(19) United States
(12) Patent Application Publication
(10) Pub. No.: US 2024/0071623 A1
(43) Pub. Date: Feb. 29, 2024

(54) PATIENT HEALTH PLATFORM

(71) Applicant: AXL Health, LLC, Chicago, IL (US)

(72) Inventors: James Richard Howard, Lago Vista, TX (US); William Ni, Huntington Beach, CA (US)

(21) Appl. No.: 18/446,141

(22) Filed: Aug. 8, 2023

Related U.S. Application Data
(60) Provisional application No. 63/374,094, filed on Aug. 31, 2022.

(51) Int. Cl. G16H 50/30; G16H 50/70

(52) U.S. Cl. CPC G16H 50/30; G16H 50/70

(57) ABSTRACT

A system that can receive non-clinical data related to an individual from one or more third party servers, and clinical data for that individual from one or more healthcare systems. The system can also pre-processes the non-clinical data and the clinical data, such as, for example, reduce the cardinality of the data and standardize the formatting of the data. The system can further project a propensity of the individual for developing at least one chronic disease by correlating the non-clinical data with the clinical data. Further, the system can identify, for each chronic disease, non-clinical factors that positively or negatively affect the propensity of the individual to develop that chronic disease, and generate a portal for display a projection for the individual to develop the chronic disease(s). At least a portion of the non-clinical risk factors can be identified by the system distilling information from secondary guidelines.

### FIELD OF DISCLOSURE

The present disclosure generally relates to a patient health platform, and, more specifically, to systems and methods for developing relationships between clinical and non-clinical information for identifying a current and/or prospective propensity of an individual to develop one or more chronic disease(s).

### BACKGROUND

Traditionally, the overall health of an individual often involves the individual occasionally visiting a primary care physician for a routine check-up or medical evaluation. Such check-ups can include the individual, also referred to herein as an end user or patient, being subjected to, and/or providing samples or specimens for, laboratory (“lab”) work, diagnostic testing, and/or analysis. For example, in at least certain instances, in connection with having lab work in the form of blood work performed, a blood sample may be taken from the individual. Such blood samples can then be analyzed or measured to provide information regarding the cells, chemicals, proteins, or other substances in the individual's blood.

The outcome of a primary care visit, including the outcome of the lab work or testing associated with that particular visit, can provide information that at least some individuals may believe provides a basis for a sense of that individual's current state of health. Yet, such information may be not captured regularly. For example, certain individual's may only annually visit a physician for a cheek-up, while other individuals may only sporadically, at best, seek medical evaluation. In such situations, the duration of time between medical evaluations can be insufficient for accurately forecast an individual's real-time propensity to develop a specific chronic disease(s), including, but not limited to, a chronic illness(es). Further, such visits may typically provide individuals with a health assessment at a particular moment in time, and capture only the consequences or indicators, and not the causes, of an individual's current health state. Moreover, the moment in time with respect to the outcome of a particular medical evaluation may not forecast future health trends for that individual, including, but not limited to, a propensity of the individual to develop a chronic disease(s).

### SUMMARY

in some embodiments, a method is disclosed herein for determining a propensity of an individual to have or develop one or more chronic diseases and output the determined propensity for display on a precision patient health platform device. The method can include receiving, by a back-end system and from one or more third party servers, non-clinical data related to the individual. The back-end system can also receive, from one or more healthcare systems, clinical data related to the individual. The back-end system pre-processes the non-clinical data and the clinical data and can project the propensity of the individual for developing at least one chronic disease of the one or more chronic diseases by correlating the non-clinical data and the clinical data, such as, for example, via identify a statistical relationship between the non-clinical data and the clinical data. The back-end system can also identify, for the at least one chronic disease, at least one non-clinical risk factor that positively or negatively affects the propensity of the individual for developing the at least one chronic disease. The back-end system can also generate a portal comprising a projection of the propensity of the individual to develop the at least one chronic disease.

In some embodiments, a lion-transitory computer readable medium is disclosed herein. The non-transitory computer readable medium can include one or more sequences of instructions, which, when executed by a processor, can cause a computing system to perform operations. The operations can include receiving, from one or more third party servers, non-clinical data related to an individual. The operations can further include receiving, from one or more healthcare system, clinical data related to the individual. Additionally, the operations can include pre-processing, the non-clinical data and the clinical data and projecting a propensity of the individual for developing a chronic disease by correlating the non-clinical data with the clinical data, such as, for example, by identifying statistical relationships between the non-clinical data and the clinical data. The operations can further include identifying non-clinical factors that positively or negatively affect the propensity of the individual for the chronic disease. Further, the operations can include generating a portal comprising a projection of the propensity of the individual to develop the chronic disease.

In some embodiments, a system is disclosed that can include a processor and a memory. The memory can have programming instructions stored thereon, which, when executed by the processor, can cause the system to perform operations. The operations can include receiving, from one or more third party servers, non-clinical data related to an individual. The operations can further include receiving, from one or more healthcare system, clinical data related to the individual. Additionally, the operations can include pre-processing the non-clinical data and the clinical data and projecting a propensity of the individual for developing at least one chronic disease of a plurality of chronic diseases by correlating the non-clinical data with the clinical data. The operations further include identifying, for each chronic disease of the plurality of chronic diseases, non-clinical factors that positively or negatively affect the propensity of the individual for that chronic disease. Further, the operations further include generating a portal comprising projections for the individual developing at least one chronic disease.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrated only typical embodiments of this disclosure and are therefore not to be considered limiting of its scope, for the disclosure may admit to other equally effective embodiments.

FIG. 1 illustrates a block diagram of an exemplary system that includes a precision patient health platform according to certain embodiments of the subject disclosure.

FIG. 2 illustrates a block diagram of at least a portion of an exemplary precision patient health platform.

FIG. 3A illustrates a block diagram depicting a non-limiting examples of correlations by the precision patient health platform of clinical data and non-clinical data.

FIG. 3B illustrates a block diagram depicting a non-limiting, example of a correlations by the precision patient health platform of clinical data, non-clinical data, and diagnoses data.

FIG. 4 illustrates a flow diagram of an exemplary method of the precision patient health platform using clinical and non-clinical data in the form of health and behavioral data, respectively, in projecting an impact of an individual's behavior on that individual's overall health.

FIG. 5A illustrates a block diagram of an exemplary system bus architecture ail computing system fir either, or both, a user device and/or a back-end computing system for the system shown in FIG. 1.

FIG. 5B illustrates a block diagram of an exemplary computing system having a chipset architecture for either, or both, a user device and/or a back-end computing system for the system shown in FIG. 1.

FIG. 6 illustrates an exemplary patient journey chart displayed on a graphical user interface (GUI) of a precision patient health platform device according to an illustrated embodiment of the subject disclosure.

FIG. 7 illustrates an exemplary interactive plot displayed on a graphical user interface (GUI) of a precision patient health platform device according to an illustrated embodiment of the subject disclosure.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Embodiments disclosed herein generally relate to a system and method for correlating clinical and non-clinical data pertaining to an individual to project a propensity of that individual for developing a chronic disease. For example, according to certain embodiments, clinical data can correspond to a first set of first categories of clinical information that may be obtained via examination, testing, or analysis by a physician or other medical related professional, including obtained via associated testing and diagnostic analysis of an individual or sample/specimen from that individual. Thus, for example, one or more of the first categories of clinical data can include for example, information obtained from laboratory work involving analysis of a blood sample from an individual. According to certain embodiments, the clinical data can be used to generate a clinical profile of an individual, which can, from generate, from a perspective of the clinical data, provide an first, clinical, indication of the health of the individual.

Unlike clinical data, non-clinical data can correspond at least to a second set of second categories of non-clinical data that can correspond to behavior, habits, tendencies, routines, lifestyle, among other information, related to the individual. Moreover, according to certain embodiments, non-clinical data, as well as the associated second categories, can correspond to certain predetermined non-clinical risk factors, and, more specifically, can provide information regarding the habits, tendencies, behaviors, and/or lifestyle of the individual. Thus, such non-clinical data can allow for the generation of a non-clinical profile, which, from the perspective of the non-clinical data, provide a second, non-clinical, indication of the health of the individual. For example, according to certain embodiments, one or more of the second categories can be based, at least in part, on non-clinical risk factors relating to current and/or historical consumption characteristics of the individual with respect to, for example, food, media, and/or products, among other risk factors that can contribute to the iron-clinical data. Further, according to certain embodiments, one or more of the second categories of non-clinical data can correspond to non-clinical risk factors such as the amount of time the individual spends, or does not spend, on social medial, social networks, and/or other time the individual spends generally in front of a screen of an electric and/or communication device, also referred to as screen time, among other sources of information pertaining to various forms of risk factors. As also discussed below, such non-clinical risk factors can also relate to one or more of the second categories of information regarding the amount of activity/inactivity of the individual, including, with respect, to exercise, travel habits, and/or recreational and/or employment activities, among other activities of the individual.

According to certain embodiments discussed herein, the first, clinical profile and the second, non-clinical profile, of the individual, and/or the associated data, can be utilized to generate a personal health profile of the individual. Additionally, as discussed below, according to certain embodiments, personal heath profiles for an individual can be customized with respect to which, if any, of the first set of categories, also referred to a first categories, and associated clinical risk factors, for clinical information and/or the second set of categories, also referred to as second categories for non-clinical data that are considered, or not considered, in the generation of the personal health profile of the individual.

According to certain embodiments, the non-clinical data can be used by the system of the subject disclosure to project a propensity of the individual for one or more, if not a plurality, of chronic diseases by identifying a statistical relationship(s) between the clinical data and the non-clinical data. In some embodiments, to assist with downstream processes of identifying statistical relationships between clinical data and non-clinical data, the system can perform various operations or transformations using the underlying clinical or non-clinical data. For example, in some embodiments, the clinical and non-clinical data can undergo a distillation process, in which the system can map or otherwise correlate the clinical and non-clinical data to known factors associated with various chronic diseases. Further, such factors to which the clinical and-non-clinical data is mapped or otherwise correlated by the system may, or may not, be similar to at least some, if not all, of the risk factors discussed above with respect to the non-clinical data.

In some embodiments, once the propensity for each of one or more chronic diseases is/are determined, the system is configured to identify those non-clinical factors of the individual that can contribute, or provide a basis for, at least a portion of the non-clinical data that positively or negatively affect the propensity of the individual for that chronic disease(s). In this manner, the system of the subject disclosure can not only project the individual's propensity for one or more chronic diseases based on the clinical and non-clinical data, but the system can also identify those risk factors that may attribute to presence and/or development of such a chronic disease(s). Further, according to certain embodiments, such identification of the risk factors that may attribute to the presence or development of the identified chronic disease(s) can be distinguished or identified by the system in terms of the relative higher significance, role, or influence, one or more of such risk factors may have in comparison to one or more less significant or influential risk factors in the presence or development of that chronic disease(s). Such identification of pertinent risk factors and/or differences in the relative impact such identified risk factors may, in comparison to other risk factors, may have on the presence or development of identified the chronic disease(s) may assist the associated individual in developing habits, behaviors, or tendencies to anticipate, prevent, and/or manage that chronic disease(s).

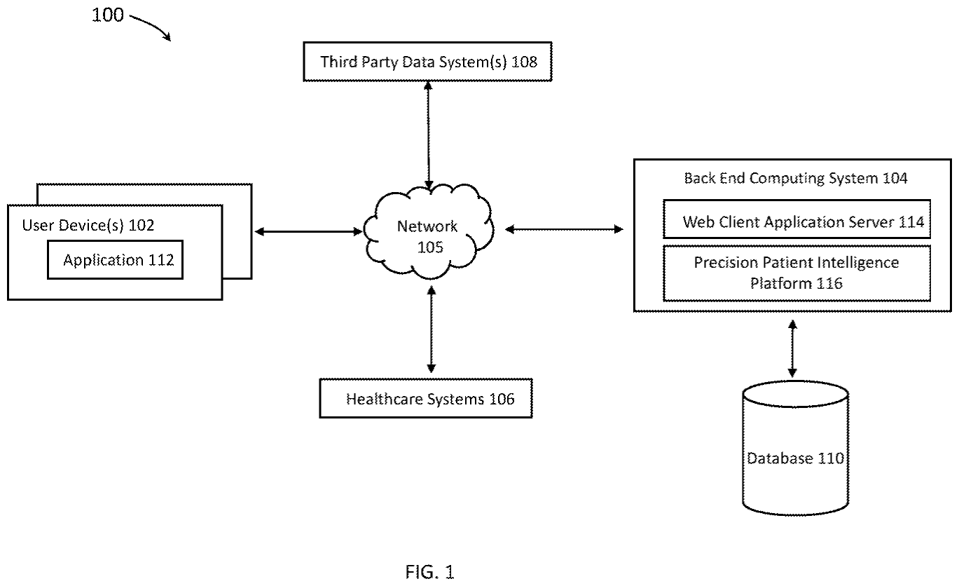

FIG. 1 illustrates a block diagram depicting an exemplary system 100 that includes a precision patient health platform 116 according to certain embodiments of the subject disclosure. The system 100 can include, and/or be communicatively coupled to via a network 105, at least one or more precision patient health platform devices 102, a back-end computing system 104, one or more healthcare systems 106, and/or one or more third party data systems 108.

The network 105 can be of any suitable type, or combinations of types, of communication protocols, including, but not limited to, individual connections via the Internet, such as cellular or Wi-Fi networks. In some embodiments, the network 105 can connect terminals, services, and mobile devices using direct connections, such as, for example, radio frequency identification (MD), near-field communication (NEC), Bluetooth™, low-energy Bluetooth™ (BLE), Wi-Fi™, ZigBee™, ambient backscatter communication (ABC) protocols, USB, WAN, and/or LAN. As at least some of the information transmitted via the network 105 can be personal or confidential, in an effort to address security concerns, one or more of these types of connections of the system 100 can be encrypted or otherwise secured. Additionally, or alternatively, as some of the information transmitted via use of the network 105 may be less personal, or not confidential, the type of communication protocol used for at least some of the network connections may be selected for convenience and/or ease of use over security.

The network 105 can include any type of computer networking arrangement used to exchange data. For example, according to certain embodiments, the network 105 may be the Internet, a private data network, a virtual private network using a public network, and/or other suitable connection(s) that enables components of the system 100 to send and receive information between the components of the system 100.

The system 100 can also include one or more precision patient health platform devices 102 that can be operated by an individual using, or being a participant for, the system 100. A variety of different devices can be utilized as the precision patient health platform device 102, including, for example, a mobile device, tablet, desktop computer, laptop, personal computing device, smartphone, or any other computing system having the capabilities described herein. The precision patient health platform device 102 can include a memory having at least one process that can be used with at least one software application (“application” or “app.”) 112. The application. 112 can be representative of an application associated with the back-end computing system 104. In some embodiments, the application 112 can be a standalone application associated with back-end computing system 104. Additionally, or alternatively, in some embodiments, the application 112 can be representative of a web browser that is configured to communicate with the back-end computing system 104. In some embodiments, the precision patient health platform device 102 can communicate over the network 105 to request a webpage, for example, from a web client application server 114 of the back-end computing system 104. For example, the precision patient health platform device 102 can be configured to execute the application 112 to access functionality of the precision patient health platform 116. For example, via the application 112, an individual can gain insights related to clinical data and/or non-clinical data, including risk factors, associated with that particular individual. The non-clinical data, risk factors, or related content that can be displayed to the precision patient health platform device 102 can be transmitted from the web client application server 114 to the precision patient health platform device 102, and subsequently processed by the application 112 for display through a graphical user interface (GUI) shown on a display or other input/output device of the precision patient health platform device 102.

The back-end computing system 104 can include a web client application server 114 and the precision patient health platform 116. The precision patient health platform 116 can be comprised of one or more software modules that can each comprise collections of code or instructions stored on a media (e.g., a memory of back-end computing system 104) that represent a series of machine instructions (e.g., program code) that implements one or more algorithmic steps. Such machine instructions can be the actual computer code one or more processors of back-end computing system 104 can interpret to implement the instructions or, alternatively, can be a higher level of coding of the instructions that are interpreted to obtain the actual computer code. The one or more software modules can also include one or more hardware components. One or more aspects of an example algorithm can be performed by the hardware components (e.g., circuitry) itself, rather than as a result of the instructions.

The precision patient health platform 116 can be configured to correlate non-clinical data with clinical data, as further discussed below, to provide correlated data. For example, according to certain embodiments, the precision patient health platform 116 can identify a statistical relationship between the non-clinical data and the clinical data that indicates a propensity of having or developing a chronic disease(s). For example, the system can identify such a statistical relationship between clinical and non-clinical data via use of historical data and/or information from secondary guidelines, as well as combinations thereof, among other information, indicating that a propensity of having certain data points, or associated values or presence of those data points, from a combination of the non-clinical data and the clinical data being associated with an individual that has or will develop/may be developing a particular chronic disease(s). Based on the correlated data, the precision patient health platform 116 can generate insights or information that can identify or otherwise describe how the risk factors associated with the non-clinical data is, or can, impact the personal health profile for that individual. Such insight or information generated by the precision patient health platform 116 can be utilized to generate, or otherwise provide a basis for determining, a strategy that the associated individual can implement to further manage and/or improve that individual's health state.

The precision patient health platform 116 can, via the network 105, be in communication with one or more healthcare systems 106 and one or more third party data systems 108. The healthcare system(s) 106 can be representative of one or more providers and/or payers associated with an end user. Thus, for example, at least some of the healthcare system(s) 106 can be associated with the physician(s), or other medical related professional(s), and associated hospitals, organizations, or entities that conduct, and/or store information regarding, medical evaluations, diagnoses, treatments, and/or medical prescriptions, among other information, of or for an associated individual(s) or patient. Thus, such healthcare systems 106 can provide information regarding the outcomes, among other information, of diagnostic analysis, testing, and measurements of the individual and associated samples or specimens. Moreover, according to certain embodiments, the healthcare system. 106 can be a source of clinical data, including with respect to at least a portion of the above-mentioned first set of first categories, for the precision patient health platform 116. However, as indicated below, according to certain embodiments, the healthcare system 106 can also be a source of at least certain non-clinical data relating to the individual.

By being communicatively coupled to one or more healthcare systems 106, the precision patient health platform 116 can receive or retrieve non-clinical and clinical data, such as, for example, healthcare related data, associated with a plurality of individuals from a healthcare system(s) 106. Exemplary clinical data can include one or more first categories of clinical data pertaining to healthcare related data such as, but not limited to, background data (e.g., family health history, age, gender, ethnic background, etc.), and/or medication data (e.g., statins, low-dose aspirin, anti-anxiety, etc.), and/or patient specific data (e.g., body mass index (BMI), weight, blood pressure, cholesterol, clinical diagnoses (e.g., diabetes, anemia, etc.)), among other data. Non-clinical data for an individual that can be attained from the healthcare system(s) 106, among other sources for non-clinical data, can include, for example, one or more second categories of data that pertain to habitual data (e.g., alcohol consumption, smoking, drug consumption, physical activity, etc.) and/or routine data (e.g., medicate refill frequency, check-up frequency, etc.), among other data that can indicate the tendencies, habits, behaviors, and/or lifestyle or lifestyle choices of the individual.

The one or more third party data systems 108 can be representative of one or more third party data aggregators. Such data aggregators can provide a tool, system, or service that collects data or information from a variety of difference sources that can be used to build the second, non-clinical profile and/or personal health profile of an individual. Further, the data aggregators of the third party data system 108 can be used to consolidate data, and particularly non-clinical data, including non-clinical data relating to risk factors, that is/arc retrieved, or otherwise obtained, from a variety of different sources into a management collection, or single set, of data. According to certain embodiments, such consolidation of data can include the organization or arrangement of data into one or more of the second categories of non-clinical data. Examples of such data aggregators can include third party services, such as, but not limited to, data collection and analytic service providers such as Epsilon™, Acxionim™, and Experian™, among others. Accordingly, via information retrieved from one or more third party data systems 108, the precision patient health platform 116 can receive or retrieve non-clinical data, including underlying data relating to associated risk factors, associated with a plurality of individuals. Exemplary non-clinical data can further include, but is not limited to, one or more second categories of non-clinical data that pertains to travel information (e.g., business travelers, leisurely travelers, etc.), event information (e.g., movie theatre purchase may mean the user bought carbonated beverages and snacks), purchase information (e.g., men's big and tall apparel), and/or food and drive purchases (e.g., fast food restaurants, vegan restaurants, etc.), among other information.

As seen in FIG. 1, the system 100 can also include one or more databases 110. According to certain embodiments, the database 100 may be part of, or communicatively coupled to, the back-end computing system 104, among other components of the system 100. Additionally, or alternatively, the database 110 can be communicatively coupled to the other devices or components of the system 100 via the network 105. Further, the database 110 can take a variety of different forms, including, for example, being a cloud based database 110. According to certain embodiments, the database 110 can store clinical and/or non-clinical data. For example, the database 110 can store data from the one or more healthcare systems 106 and/or the one or more third party data systems 108, including, for example, at least a portion of either or both of the above-mentioned first and/or second categories pertaining to clinical data and non-clinical data, respectively.

The database 110 can be communicatively coupled to the back-end computing system 104, and, moreover, the precision patient health platform 116 such that the precision patient health platform 116 can retrieve the clinical data and/or non-clinical data that is stored on the database 110. Using at least at least some of clinical and/or non-clinical information stored on the database 110, among other information, the precision patient health platform 116 can build the first, clinical profile, the second, non-clinical profile, and/or the personal health profile for each individual. One or more, if not all, of such profile can, for example, be used by at least the precision patient health platform 116 in downstream processes. Thus, according to certain embodiments, the precision patient health platform 116 can be configured to generate, for an individual, either, or both, a separate first, non-clinical profile and a separate second, non-clinical profile. Additionally, or alternatively, according to certain embodiments, in addition to, or in lieu of, having one or both of the first, clinical profile and the second, non-clinical profile, the precision patient health platform 116 can be configured to generate a personal health profile of an individual. According to certain embodiments, using a user interface or other input/output device of the precision patient health platform device 102, an individual can select which profile(s) for that individual. (e.g., first, clinical profile, second, non-clinical profile, and/or personal health profile) are, at that time, to be displayed on a display of the precision patient health platform device 102. Additionally, the personal health profile may be a combination of at least portions, if not the entirety of, the first, clinical profile and the second, non-clinical profile. For example, according to certain embodiments, the personal health profile may be selectively customizable based on the data from, or underlying factors associated with, either or both the first, clinical profile and the second, non-clinical profile that is utilized, or not utilized, in connection with generating the personal health profile. Thus, for example, according to certain embodiments, data corresponding to the one or more of the first categories of clinical data, and/or to one or more second categories of non-clinical data can be selected for inclusion, and/or, alternatively, for exclusion, in use by the precision patient health platform 116 in generating a personal health profile for an individual. Such selective inclusion and/or exclusion of first categories of clinical data can also be utilized in connection with the precision patient health platform 116 generating an individual's clinical profile, and/or with respect to the inclusion and/or exclusion of second categories of non-clinical data used in connection with the precision patient health platform 116 generating an individual's profile.

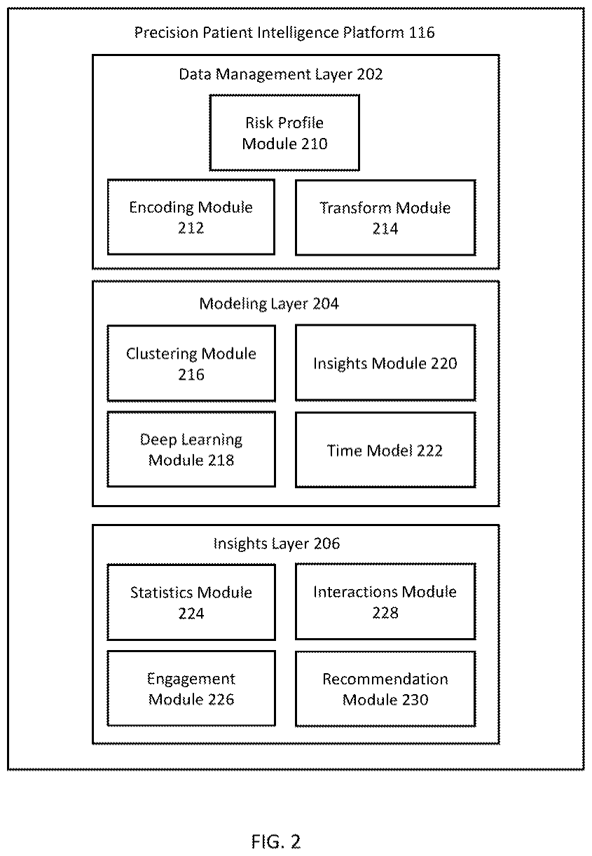

FIG. 2 illustrates a block diagram of at least a portion of an exemplary precision patient health platform 116, according to certain illustrated embodiments. As shown, the precision patient health platform 116 can include one or more layers, including, for example, a data management layer 202, a modeling layer 204, and/or an insights layer 206, among other layers. The data management layer 202 can be configured to pre-process either, or both, the clinical and non-clinical data for input to the modeling layer 204. Such pre-processing by the data management layer 202 can include, for example, formatting, reformatting, and/or refinement of the clinical and non-clinical data, and/or addressing errors in the content, format, or style of the clinical data and/or non-clinical data. Thus, for example, the data management layer 202 can generally be configured to cleanse or otherwise format clinical and non-clinical data in a manner that prepare the clinical and non-clinical data for use by other portions of the precision patient health platform 116 and/or in connection with other, downstream processes performed using the precision patient health platform 116, among other components of the system 100. As shown in FIG. 2, according to certain embodiments, the data management layer 202 can include a risk profile module 210, an encoding module 212, and/or a transform module 214.

The risk profile module 210 can be configured to digest secondary guidelines produced or derived by one or more external source. Such secondary guidelines can be retrieved or accessed by the system 100 and/or the precision patient health platform 116 via, for example, a connection to the Internet via the network 105. Further, such external sources can include, but are not limited to, subscription based publication services, including, for example, PubMed™ among others, from the external source(s), can also be specific to one or more first or second categories pertaining to clinical and non-clinical data, or associated risk factors, respectively, while also being non-specific, or directed specifically, to the particular individual, such as patient, for which a profile is being generated. For example, according to certain embodiments, the secondary guidelines could relate to information from scientific literature providing information used by the precision patient health platform 116 to link certain categories of clinical data and certain categories of non-clinical data to risk factors for a personal heath profile relating the existence, or potential development, of a cardiovascular disease.

According to certain embodiments, such secondary guidelines can be published literature or journals that may be disseminated from an author or entity that is unrelated to the physician or medical professionals that were involved with activates from which clinical data was generated or collected. Thus, for example, according to certain embodiments, such secondary guidelines can be provided from publically available scientific literature, including, but not limited to, scientific journals, papers, pamphlets, brochures, technical reports, books, aid/or presentations developed in connection with scientific research and/or testing by a researcher(s), research organization(s), field expert(s), and/or academic(s), among other experts and professionals in the medical field.

Additionally, according to certain embodiments, such secondary guidelines can, before use by the precision patient health platform 116, undergo a screening process that can evaluate the credibility or reliability of corresponding publications or literature that can potentially be utilized by the precision patient health platform 116 as a secondary guideline(s). Such screening, which can be performed via the data management layer 202, among other portions of the system 100 and/or precision patient health platform 116, and can evaluate whether the proposed publication or literature satisfies certain criteria, including, for example, but not limited to, criteria with respect to the credentials of the source, author, and/or researcher(s) of the publication or literature, and/or whether the publication or literature has satisfied a peer or industry review process, among other filtering criteria. Thus, such criteria used in evaluating publications or information for use as secondary guidelines can be directed to seeking to confirm the reliability, trustworthiness, acceptance, and/or credibility of the associated underlying information.

The risk profile module 210 can automatically extract and derive information for use by the precision patient health platform 116, including with respect to generating clinical, non clinical, and/or personal heath profiles or associated evaluating certain associated risk factors, in a variety of different manners. For example, according to certain embodiments, the risk profile module 210 can utilize one or more natural language processing techniques to derive information from secondary guidelines that can provide information, or one or more basis/bases for identifying and/or evaluating one or more risk factors associated with one or more chronic diseases. Using a specific example, for cardiovascular disease, the risk profile module 210 can digest or otherwise use secondary guidelines in the form of scientific literature to determine that non-clinical risk factors or second, non-clinical categories associated with (1) habits relating to caffeine intake can have a negative impact on cardiovascular health, and (2) engagement in fitness training can have a positive impact on cardiovascular health. Accordingly, from such information, the risk module 210 can utilize technics to learn or otherwise identify (e.g. via artificial health (AI) machine learning techniques) that caffeine and fitness training can be two risk factors that can be associated with cardiovascular health. According to such an example, the risk factors utilized in connection with at least portions of a non-clinical and/or personal health profile for an individual pertaining to cardiovascular health, among other specific medical conditions, can include, among other risk factors, a first risk factor, such as, for example, extent of caffeine intake, that can negatively correspond to that specific medical condition, and/or another, or second factor, such as an extend of fitness training, that can positively correspond to that specific medical condition. While the foregoing non-limiting example is discussed with respect to first and second risk factors, as well as in the context of a medical condition relating to cardiovascular health, any number, or combinations, of positive and negative health factors can be utilized in connection with medical conditions in addition to, or in lieu of, cardiovascular health.

The encoding module 212 can be configured to encode an input data, such as, for example, clinical data and non-clinical data, among other data utilized by, or provided to, the precision patient health platform 116, into a standardized format(s). Such encoding of the input data can assist in downstream processing of the input data. In some embodiments, the encoding module 212 can encode or transform the input data (e.g., the clinical data and non-clinical data) into industry standard formats, including standardized format used in the health care industry, including, for example, a unified Fast Healthcare Interoperability Resources (FHR®) standard, among other formats. Such use of standardized formatting, as provided via operation of at least the encoding module 212, can be configured to facilitate the interoperability of data used and/or generated by the precision patient health platform 116 with other healthcare information systems. Thus, the encoding module 212 can be configured to format the input data in a manner that complies with generally consistent structures and sets of protocols for the exchange, integration, sharing, and retrieval of electronic health information by, and/or between the precision patient health platform 116 and other healthcare information systems.

The transform module 214 can be configured to map, including associate or link, non-clinical data for an individual to one or more of the above-mentioned second, non-clinical categories and/or to one or more of the associated non-clinical risk factor(s) of one or more of those second categories. Thus, for example, according to certain embodiments, such mapping of the non-clinical data of an individual can include mapping the non-clinical data to one or more risk factors that were derived or otherwise transformed from one or more secondary guidelines, including, for example, peer reviewed scientific literature, by the risk profile module 210, as discussed above. According to certain embodiments, embodiments, the transform module 214 can be configured to perform one or more sub-processes, including, for example, two sub-processes, such as, for example, a distillation process and a refinement process.

During the distillation process, the transform module 214 can be configured to distill, or reduce the cardinality, of the non-clinical data and/or the clinical data. For example, such distillation can involve the transform module 214 distilling clinical data and/or data down from a relatively large feature space, or data points, to a smaller feature space, or data points. For example, with respect to non-clinical data, a certain number of data points can be generated for an individual every day that can correspond to any number of categories of non-clinical data (e.g., websites the individual viewed, stores or other places the individual visited, purchases, etc.). Thus, non-clinical data collected on daily, weekly, monthly, yearly, etc. basis can result in relatively large data sets potentially having tens to hundreds of thousands of non-clinical data points. Thus, the transform module 214 can perform a distillation process in which transform module 214 can map the behavioral data points to non-clinical categories and/or risk factors that can be indicative of a behavior or other non-clinical characteristics of the individual that may positively, or negatively, affect the a propensity for that individual to have, or have a risk of developing, one or more chronic diseases or may be tied to a health risk, and/or a health equities risk that can be associated with equitable access to health related resources and opportunities, as may, for example, be defined by the information derived from the secondary guidelines, including, for example, scientific literature.

Using a specific example, an individual's non-clinical data can include several purchases relating to caffeinated drinks from a particular retailer or seller, including, but not limited to, coffee purchases from a particular retailer, which, for purposes of this example, can be a store, retailer, franchisee, and/or entity name “Retailer1”. Thus, according to certain embodiments, the precision patient health platform 116 can be configured such that specific item(s), such as, for example, coffee, can be identified by, or otherwise mapped to, purchases made by the individual from, and/or determined to have occurred based on an identification of, the retailer, in this example, Retailer1. Thus, while the secondary guidelines, including scientific literature, may not directly map Retailer1 to cardiovascular disease, the platform 116 may derive the type or characteristics of the item(s) purchased by the individual from an identification of the associated retailer, such as, for example, derive that one or more purchases of coffee were made by the individual based on an indication of the individual having made a purchase from, or at, Retailer1. Accordingly, in this example, by making a purchase at/from Retailer1, the transformation module 214 can map a purchase and/or consumption of coffee by the individual to one or more risk factors related to clinical and/or nonclinical categories.

Further, as previously discussed, data mapped to a risk factor(s) that were, for example, derived by the risk profile module 210, can correspond an indication of a negative, or, alternatively, a positive propensity to have, or not have, or develop one or more chronic diseases. Thus, in the foregoing example, consumption of coffee by the individual, which, again, may be determined based on an indication of a purchase by the individual from Retailer1, can have a negative effect on a clinical category(ies) relating to identifying the individual having, or developing, a chronic disease in the form of cardiovascular disease. Similarly, such a risk factor, in this example, consumption of coffee, can also be mapped, either positively or negatively, to several other categories and/or chronic diseases, also referred to as health care issues, such as, but not limited to, other clinical categories relating to, ands the chronic disease(s) of, dementia and/or gallstones, among others. Accordingly, by mapping the individual's purchases of “coffee” and/or inning such “coffee” purchases based on where, or from who, including retailer, that the purchases occurred, the transform module 214 can utilize a single risk factor to map the consumption of coffee by the individual to one or more chronic illnesses (e.g., cardiovascular disease, dementia, gallstones, etc.). Accordingly, in this manner, the transform module 214 can distill or map the non-clinical data to risk factors, as may be derived via use of the secondary guidelines, associated with one or more clinical and/or nonclinical categories in a manner that can be used by the precision patient health platform 116 to derive a clinical, non-clinical, and/or personal health risk profile for that individual that can be indicative of risk that individual may have, or for developing, one or more chronic diseases.

The transform module 214 can also be configured to perform processes for clinical data that are similar to those discussed above with respect to non-clinical data. For example, a variety of different types of health codes, such as, for example codes utilized by the International Classification of Diseases (“ICD”) 10, among other codes, systems, or classifications can be utilized to describe, characterize, or code one or more, or a set, of patient attributes, including, for example, diseases or health conditions, such as, but not limited to, treatments and/or symptoms. Given the potentially relatively large number of such health codes, including sets of health codes, the transform module 214 can be configured to consolidate such health codes into higher order semantic categories that describe, for example, the afflicted organ aid/or overall patient health. In some embodiments, such health codes can be consolidated into higher order semantic categories using ontologies or hierarchies from at least certain secondary guidelines, including, but not limited to, scientific literature. For example, ICD health codes can be consolidated into chapter, or sub-chapter, semantic representations.

Moreover, as ICD health codes are part of a relatively detailed and complex system used to categorize diseases and health conditions. Thus, according to certain embodiments discussed herein, the transform module 214 can consolidate the LCD health codes into broader categories known as chapters or sub-chapters for easier analysis. For example, individual ICD health codes can be grouped into broader categories for ease of understanding and/or analysis. According to certain embodiments, ontologies or hierarchies from the secondary guidelines, including scientific literature, can be utilized by the transform module 214 as frameworks for categorizing knowledge or information provided by such secondary guidelines. Such grouping of information can be structured in, a manner that can, for example, be similar to that used in other fields, such as, for example, bioinformatics. Accordingly, with such an approach, such framework, as derive by, for example, the transform module 214, can be used to determine the relevant higher order categories into which individual health codes, or associated sets of health codes, are to be grouped.

During the refinement process, the transform module 214 can be configured to refine the distilled non-clinical data such that the distilled non-clinical data is more amenable to downstream processes. In some embodiments, the refinement process can include the transform module 214 automatically cleansing data from outliers and other erroneous entries. In some embodiments, the refinement process may include transform module 214 normalizing the distilled non-clinical data (e.g., mean and variance normalization). For example, the above-discussed example referenced a determination that coffee was purchased based on an indication of a purchase from Retailer1, as may be indicated, for example, a charge or credit card statement, among other sources of purchase history information. However, in certain instances, such a charge or credit card may be shared among different people, such as, for example, multiple family members. In such a situation, the refinement process may seek to determine if the purchase indicated by the purchase history was made by, or for, the individual for which the health assessment is being generated by the precision patient health platform 116. For example, the type of retailer or purchase, and/or geo-tracking, among other information, can be utilized in an attempt during the refinement process to at least attempt to identify whether the purchase, was, or was not, made by or for the subject individual, and thus whether to utilize that purchase in connection with a positive/negative risk factor(s) in the health assessment for that individual that is being generated by the precision patient health platform 116.

The modeling layer 204 can be configured to perform various data operations on the data processed by data management layer 202. As shown, and as discussed below, the modeling layer 204 can include a clustering module 216, a deep learning module 218, an insights module 220, and/or a time model 222.

The clustering module 216 can be configured to analyze clinical data and/or non-clinical data for the individual that is the subject of the health assessment being performed by the precision patient health platform 116 to group similar individuals, or groups of individuals, based on their non-clinical data and/or clinical data. For example, the clustering module 216 can utilize one or more clustering techniques to identify or group individuals according to their correlated similarities in their clinical, non-clinical, and/or personal heath profiles, such as, similarities in the individuals' or groups' underlying non-clinical data and/or clinical data. For example, according to certain embodiments, the clustering module 216 can utilize a k-means clustering algorithm to cluster similar individuals or groups of individuals based on a variety of clinical and/or non-clinical data points for those individuals and/or groups.

The deep learning module 218 can be configured to identify one or more chronic diseases, including chronic illnesses, for which an individual is at risk of having or developing based on the clinical, non-clinical, and/or personal heath profile(s) of that individual, and/or based on the associated underlying data points and/or determined risk factors. For example, based on the refined clinical and/or non-clinical data points, the deep learning module 218 can be trained to project a propensity of an individual either having, or developing, a chronic disease. According to certain embodiments, the deep learning module 218 can project or predict a propensity of an individual to having, or develop, a plurality of different chronic diseases, such as, but not limited to, cardiovascular disease, dementia, diabetes, and the like. According to certain embodiment, to generate such a prediction(s), the deep learning module 218 can be trained to correlate the refined non-clinical data with the refined clinical data. According to certain embodiments, such correlation of the non-clinical data with the clinical data can be based, or guided at least in part, on the information distilled from the secondary guidelines, including, for example, the scientific literature, among other information.

In some embodiments, the deep learning module 218 can utilize a gradient boosted tree model that may be trained or optimized to correlate the refined non-clinical data with the refined clinical data. According to certain embodiments, the deep learning module 218 can utilize a neural network architecture that can be trained or optimized to correlate the refined non-clinical data with the refined clinical data. To tune the model utilized by the deep learning module 218, the deep learning module 218 can undergo a supervised training process. For example, during training, the deep learning module 218 can be provided with inputs that can include, but are not limited to, a variety of input information regarding the clinical and non-clinical data for a plurality of individuals or groups of individuals, as well as information regarding the chronic disease(s) such individuals or groups have and/or have developed. In this manner, deep learning module 218 may be trained to learn the likelihood of a user having a chronic illness based on their health and behavioral data.

Moreover, prepared data can be fed into the deep learning module 218 and/or the model utilized by the deep learning module 218 from which the deep learning module 218 can be trained, including, for example, identify/learn patterns and associations. Thus, according to certain embodiment, predictions generated by the model regarding an individual(s) having, or developing, chronic diseases can be compared to known information regarding known chronic disease information for that/those individual(s). From such a comparison, to the extent there is a difference, or error, between the prediction outputted via use of the model of the deep learning module 218 and the known information, that difference/error can be utilized by the deep learning module 218 to further adjust, or modify, the model used by the deep learning module 218, including, for example, update the previously identified pattern. This learning process is repeated at least until the predictions outputted by the model of the deep learning module 218 more accurately matches the actual data, or otherwise is within a predetermined threshold or accuracy rate. Such an approach thus trains the model of the deep learning module 218 to estimate the likelihood of an individual having, or developing, a chronic disease on that individual's clinical and/or non-clinical data.

The insights module 220 can be configured to interpret outputs generated by the deep learning module 218. For example, the insights module 220 can be configured to interpret the outputs generated by the deep learning module 218 to identify features, including risk factors, among other information that may be part of the individual's clinical, non-clinical, and/or personal heath profiles, that are, or may be, most relevant or influential to a given prediction of a person having, or developing, a chronic disease. Using a specific example, the insights module 220 can deter that an individual's alcohol use, among other non-clinical data, in view of information from the individual's clinical data, is a primary contributor, if not the largest potential contributor, to that individual's propensity for having, or developing, cardiovascular disease. Accordingly, the output of the insights module 220 can provide an individual with an indication of the specific factors, based on specific information, and/or customized, for that individual, that can contribute (negatively or positively) to specific chronic diseases for which that individual has a propensity for having or developing. In some embodiments, the insights module 220 can utilize Shapley value regression techniques to determine the importance of each factor that contributes to the output generated by deep learning module 218.

The time model. 222 can be configured to model time-varying non-clinical factors to estimate future changes in persona and trajectories of user engagement and chronic disease management. For example, if it is determined that an individual has a relatively high propensity for cardiovascular disease, and that alcohol use is one of the main contributors to that projection, the time model 222 can model how a reduction in alcohol use can affect the individual's projected outcome with respect to the development and/or progression of cardiovascular disease. In some embodiments, the time model 222 can estimate the aforementioned future changes through autoregressive forecasting of clinical and non-clinical elements. In some embodiments, the autoregressive forecasting can be accomplished by a deep learning approach that is similar to that discussed above with respect to the deep learning module 218, including with respect to use of a neural network architecture and the refinement of the model and associated pattern identification/analysis utilized for the time model. 222. Thus for example, the model, utilized by the time model 222 can be a self-learning model that is trained via use of prepared data relating to historical time series of actions by a plurality or groups of individuals, including associated non-clinical data for those individuals, as well as associated data relating to the development and/or progression of a chronic disease(s). Further, similar to the above-discussed deep learning module 218, information received by the time model. 222 can be utilized to compare actual outcomes with predictions generated by the model of the time 222, and the associated differences can be utilized to improve the model and/or identified patterns of the model used by the time model 222. Further, according to certain embodiments, the architecture of the model utilized by the time model 222 can be designed to explicitly account for temporal dynamics. For example, in the case of a neural network embodiment of the autoregressive forecasting model, recurrent architectures (e.g. Long Short-Term Memory Networks, Gated Recurrent Units) and transformer architectures may both be used. In some embodiments, learning schedules and hyper parameter settings may be adaptively derived to optimize the performance of the model for the forecasting tusk.

The insights layer 206 can be configured to provide, including communicate, at least a portion of the insights generated by the modeling layer 204 to the individual, including, but not limited to, modeling layer 204 one or more of the interpretations generated by the insights module 220. As shown, and as discussed below, according to certain embodiments, the insights layer 206 can include statistics module 224, engagement module 226, interactions module 228, and recommendation module 230.

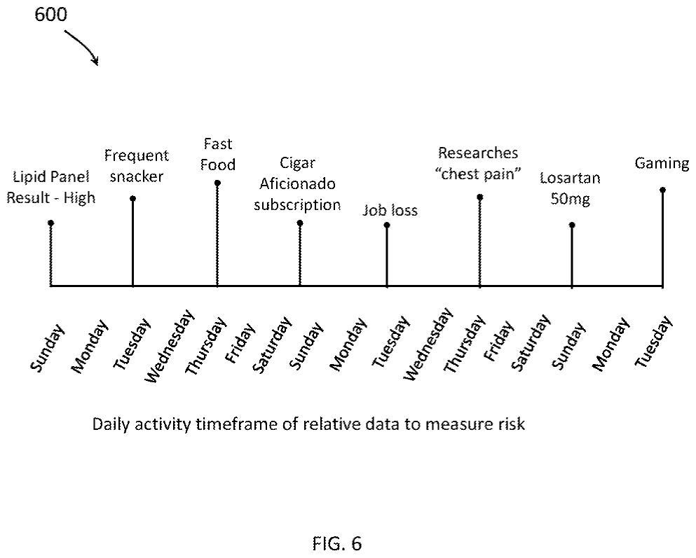

The statistics module 224 can be configured to utilized outcomes generated by the modeling layer 204 in an manner that can, at least statistically, provide observations regarding the subject individual, including, but not limited to, the personas, engagement, and management of the individual in a variety of manner, such as, for example, via one or more intuitive charts and plots, as shown, for example, by at least FIG. 6. In some embodiments, the form of the observations (e.g., the type of plot used) can be determined through the statistics module 224 generating a set of deterministic and probabilistic analyses of the observations that were outputted by the modeling layer 204. For example, a candidate set of plots, including, for example, bar, box and whiskers, and/or line plots, among others, can be mapped to observations outputted by the modeling layer 204 as a function of the type of observation (e.g., text, numerical, categorical, etc.). The selected type of plot to utilize among the candidate set of plots for a given observation can be determined in a variety of manners, including, for example, through a statistical analysis of the characteristics of the observation(s). In some embodiments, the function, including, for example, the algorithm, used by the statistics module 224 to determine the plot type, or manner of mapping, based the statistical characteristics of the observation data can be determined heuristically. For example, statistics module 224 can generate a candidate plot for the individual in the form of a patient journey chart that can be based on the information outputted by the modeling layer 204 anchor underlying clinical or non-clinical data for that individual, among other stored information about that individual that indicates the propensity for having or developing a chronic disease(s).

For example, FIG. 6 illustrates an example patient journey chart being displayed on an exemplary graphical user interface (GUI) 600 of a precision patient health platform device 102. As shown, the depicted patient journey chart can be present information such as the associated individual's habits during a specific time frame, such as, for example, on a daily basis that may affect that individual's overall health and/or with respect to one or more chronic diseases (e.g. cardiovascular disease). For example, with respect to the time frame shown in FIG. 6, the modeling layer 204, among other portions of the system 100, has identified certain days at which the activities, or risk factors, of the individual can impact the individual's health, such as, for example, during a first week, engagement in “Frequent snacker” activities/behavior on a Tuesday, eating “Fast Food” on. Thursday, and an indication of being a smoker, as seen by the system 100 detecting a purchase by the individual in the form of a “Cigar Aficionado subscription” on Sunday. The illustrated time frame shown in FIG. 6 also includes activities/behavior during a second week that can impact the individual's health, including a “Job loss”, electronic searching, including via the worldwide web or internet, of information regarding “chest pain”, and electronic gaming activities. The illustrated patient journey chart can further provide information regarding clinical data identified by the modeling layer 204, among other portions of the system 100, that can also be a risk factor in terms of the of the overall health of the individual and/or with respect to the propensity of the individual developing one or more chronic diseases. For example, as seen in the example shown in FIG. 6, the patient journey chart includes clinical data in the form of results of lab work on the patient, and, more specifically, lab work that has resulted in the outcome on Monday of a “Lipid Panel Result . . . High”. The illustrated time frame shown in FIG. 6 also includes an indication of a single instance of the individual taking 50 milligrams (mg) of a medication, which may provide a non-clinical information in the form of an indication of the individual not regularly taking medication, as prescribed.

The engagement module 226 can be configured to perform a set of outreach activities customized to one of end goals of the subject individual. Including, for example, end goals set or predetermined by the individual or otherwise learned by the system 100, including via artificial health or machine learning capabilities of the system 100. For example, the engagement module 226 can understand, including learn to understand, one or more habits of the individual in a manner that can drive engagement between the system 100 and the individual. In some embodiments, the engagement module 226 can perform outreach to the individual through a machine learning algorithm. For example, given the goals of the individual, the engagement module 226 can be configured to recommend one or more of the: (1) modality of engagement with the individual. (e.g., email, SMS, etc.), (2) message style (e.g., serious, or friendly), and for (3) message time (e.g., morning or evening). In some embodiments, the machine learning model that powers engagement module 226 can be a neural network model that may be initially trained using observational data collected regarding previous engagement with other individuals or groups of individuals, and updated as new data from prospective populations are collected.

The interactions module 228 can be configured to allow an individual the ability to manipulate, including customize, in real time information provided by the insights layer 206 for display on the GUI 600. For example, the interactions module 228 can allow the individual to, via the GUI 600 displayed cm the display of the precision patient health platform device 102 to alter the display or format of information shown in the GUI 600, such as, for example, click on and/or zoom on the displayed information, and/or control or modify the content of the displayed information, including, for example, filter the information and/or search across various data related to the individual.

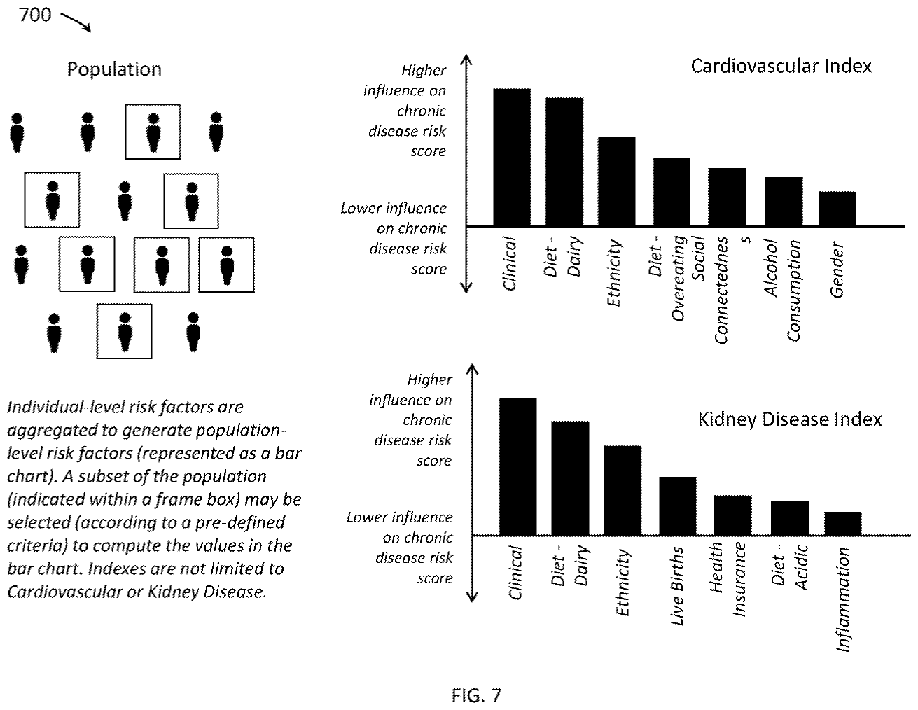

For example, FIG. 7 illustrates au exemplary interactive plot being shown on an illustrated GUI 700 of a precision patient health platform device 102 according to certain embodiments of the subject disclosure. The illustrated exemplary interactive plot includes information related to a cardiovascular disease index, a kidney disease index, and a population index. Moreover, as indicated by FIG. 7, individual-level risk factors are aggregated to generate population-level risk factors, which can be represented as a bar chart. A subset of the population, which in FIG. 7 is indicated within a frame box in the illustrated population index, can be selected, according to a pre-defined criteria, to compute the values in the bar chart. Moreover, as seen, the illustrated cardiovascular index includes several risk factors, as well as indications of the relative influence, as indicated by a bar chart, those different risk factors for the subject individual has on the propensity on the individual developing cardiovascular disease. For instance, in the illustrated example, the cardiovascular disease index indicates the following risk factors: “Clinical”, “Diet-Dairy”, “Ethnicity”, “Diet-Overeating”, “Social Connectedness”, “Alcohol Consumption”, and “Gender”. As seen by the associated bar graph, the influence of the identified risk factors for developing cardiovascular disease are ranked in the cardiovascular index in descending order from left to right order. Similarly, the illustrated kidney disease index includes several risk factors, as well as indications of the relative influence, as indicated by a bar chart, those different risk factors for the subject individual has on the propensity on the individual developing kidney disease. For instance, era the illustrated example, the kidney disease index indicates the following risk factors: “Clinical”, “Diet-Dairy”, “Ethnicity”, “Live Births” “Health Insurance”, “Diet—Acidic”, and “Inflammation”. Again, as seen by the associated bar graph, the influence of the identified risk factors for developing kidney disease are ranked in the cardiovascular index in descending order from left to right order. Further, while FIG. 7 illustrates limited to cardiovascular or kidney disease indexes, addition or other indexes can be utilized. Moreover, such indexes are not limited to cardiovascular or kidney disease indexes

The interactions module 228 can be configured to allow an individual to, for example, select clinical, non-clinical data for a cohort or population of individuals or groups to inspect through a filter, which the individual can select and/or customize. Moreover, the individual can filter information based on one or more parameters, which may be predetermined and selectively utilized by the individual. For example, certain filters can correspond to ages, genders, and/or ethnicities of the cohort or population for which information is being searched. For example, the interactions module 228 can be configured to allow an individual to filter searchable information based on the age and gender (e.g., age >50 AND male) of the cohort or population for which data is being searched. Through such selective filtering of information, as provided by the interactions module 228, the precision patient health platform 116 can utilize one or more of the above-discussed data management layer 202, modeling layer 204, and/or insights layer 206 to generate new insights.

The recommendation module 230 can be configured to generate and share recommendations for the next best actionable steps that the subject individual can perform. For example, based on the outcomes and/or determinations made using the modeling layer 204, including, but not limited to, from the deep learning module 218, outputs from the engagement module 226, and/or identified or provided goals of the individual, the recommendation module 230 can generate one or more corresponding actions from a set of potential actions. Further, according to certain embodiments, the risk profile generated for the individual, which can, for example, comprise one or more of a clinical, non-clinical, and/or personal health risk profile, can be utilized by the recommendation module 230 to identify one or more suggested recommendations with respect to actionable steps the individual can undertake to improve that individual's overall health. The recommended actions or steps can be based on a variety of criteria, including for example, the specific, or combination, of identified chronic diseases, and the current health, condition, or age of the individual, among, other criteria or factors. Further, the recommended actions or steps can relate to relate to a variety of different actions that can be taken by the individual, including with respect to exercise, diet, and/or prescriptions, and/or lifestyle choices, among other actions. According to certain embodiments, the set of potential actions from which the recommendation module 230 utilizes in deriving one or more recommendations, as well as the basis for the selected recommendations for the user, can be based on information derived by the system 100 from the secondary guidelines, as discussed above.

According to certain embodiments, the recommendation module 230 can utilize machine learning, and moreover, a neural network model that may be initially trained using data at least from secondary guidelines. Moreover, the recommendation module 210 can utilize machine learning to identify, from information distilled by the system 100 from the secondary guidelines, one or more recommended action(s) for particular chronic disease or collection of chronic diseases. Additionally, or alternatively, such machine learning can include collected data obtained for a number and/or groups of individuals that can indicate actions taken, and not taken, by those individuals/groups to address the development, and/or propensity to develop, one or more specific chronic diseases, and the outcome such actions had for those individuals in the management and/or prevention of such a chronic disease(s). Additionally, the models developed, and/or patterns identified, by use of such machine learning by the recommendation module 230 can be generally continuously refined or adjusted using comparisons of actual outcomes of taken particular actions by individuals/groups in connection with the prevention and/or management of chronic diseases and the recommendations that are, or have been, generated by the model used by the recommendation module 230.

Additionally, according to certain embodiments, the recommendations selected by the recommendation module 230 can include ranking the risk factors that are attributable to the determined risk profile for the individual, including a risk profile that is based on at least one, or more of the above-identified clinical, non-clinical, and/or personal health risk profiles.

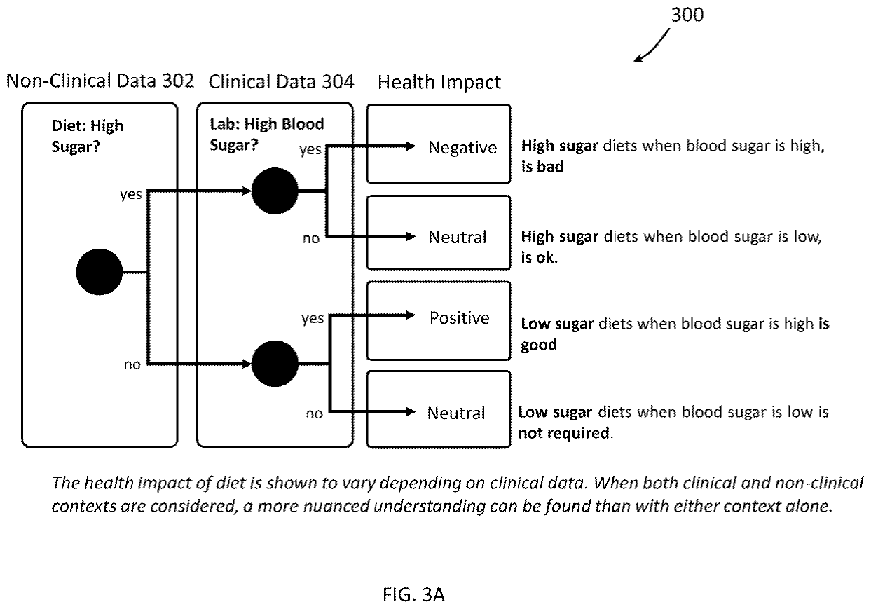

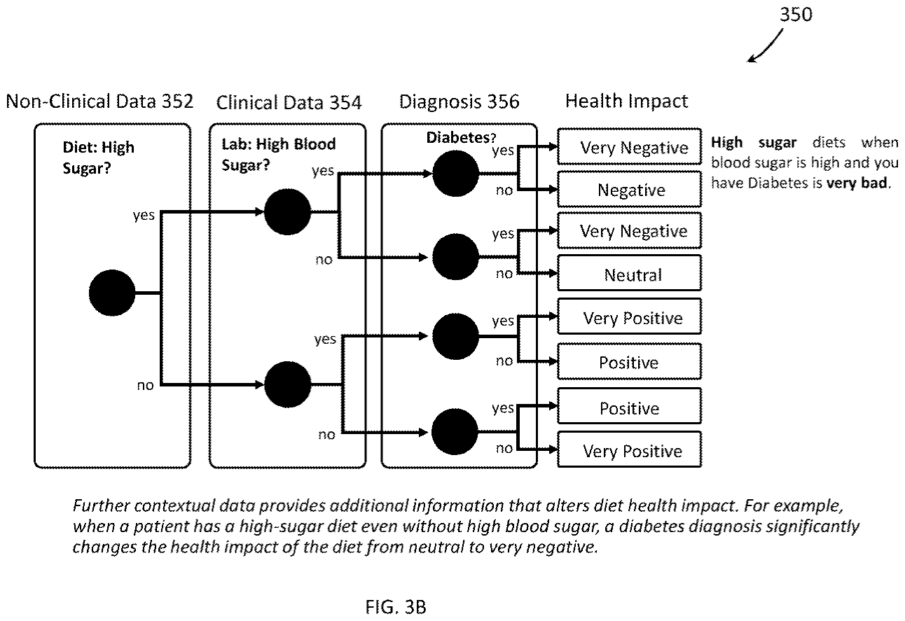

FIG. 3A illustrates a block diagram 300 depicting an example of correlations of the precision patient health platform of non-clinical data 302 and clinical data 304, according to certain exemplary embodiments. Moreover, FIG. 3A provides an example of the health impact of a diet, as can be derived from non-clinical data, varying depending on clinical data. As seen, when both clinical and non-clinical contexts are considered, a more nuanced understanding can be found than with either context alone. Thus, the behavioral data 302 in the example illustrated in FIG. 3A corresponds to an aspect of the dietary habits or tendencies of the subject individual, and, more particularly non-clinical data for a risk factor related to sugar consumption. Moreover, in this example, the non-clinical data is directed to determining whether the subject individual does, or does not, have a diet that is high in sugar consumption. As previously discussed, such non-clinical data relating to sugar consumption can be derived in a variety of manners. For example, as previously discussed, according to certain embodiments, the non-clinical data can be deduced by data management layer 202. For example, such information can be derived from information directly inputted by the individual regarding the diet and/or lifestyle, among other information, of the individual. Such information can also be indirectly obtained, and/or supplement directly obtained information, for the individual, including, for example, by reference to the stores and/or retailers from which the individual made purchases, including, for example, from purchases from stores that may be generally identified as being associated with relatively high sugar content foods or treats. For example, an indication of consumption of foods relatively high in sugar by the subject individual can be based on identifying transactions or purchases by that individual from an ice cream store(s) or candy store(s). According to such an example, the transform module 214 can map such transactions to one or more non-clinical data categories or risk factors relating to sugar consumption and/or high sugar consumption.

Further, the system 100 can identify the type of clinical data 304 that is to be used by the system 100 in connection with a correlation involving non-clinical data, including identifying which risk factor(s) and category(ies), or which associated data points, of clinical data are to be used in the correlation based on the type of non-clinical data 302 being used, and/or vice versa. According to certain embodiments, the particular non-clinical data and clinical data, or associated underlying data points, selected for use in the correlation involving the non-clinical data and the clinical data can be based on a variety of criteria, including, but not limited to, the information derived from the secondary guidelines, including scientific literature, as previously discussed. Thus, in the example presented in FIG. 3A, as the identified non-clinical data relates to sugar consumption, the clinical data 304 can similarly relate to types of clinical data that may reflect measured information that can provide an indication of regarding sugar consumption by the subject individual. Thus, in this example, the correlation utilizes clinical data in the form of blood sugar levels, as may be determined via measurements or analysis of a blood sample from the individual, and the previously mentioned non-clinical data 302.

Continuing with the example provided in FIG. 3A, in operation, the deep learning module 218 can correlate the non-clinical data 302 with the clinical data 304 to project a health impact for the subject individual, which can include an indication of the individual having or developing a chronic disease. Thus, in this illustrated example, as shown, the system 100 can derive, including determine using at least information obtained by secondary guidelines, including scientific literature, the correlation certain non-clinical data 302 and certain clinical data 304 has on a heath impact of the individual. For example, as seen in FIG. 3A, the system 100 can correlate non-clinical data 302 indicating an individual has a high sugar diet, and the clinical data 304 indicating analysis of a blood sample that indicates the individual has high blood sugar, with negative health impact. Thus, the correlation derived by the system. 100 can indicate that high sugar diets when blood sugar levels are high is bad for the health of the individual. Similarly, as also shown in FIG. 3A, the system 100 can derive a correlation between non-clinical data 302 for an individual that indicates a high sugar diet and clinical data for that same individual that indicates a lack of high blood with a neutral health impact for the individual. Thus, the correlation derived by the system MO can indicate that high sugar diets when blood sugar levels of the subject individual are low is alright or line in terms of the health of the individual.

On the other hand, if the non-clinical data 302 indicates the individual does not have a high sugar diet and the clinical data shows high blood sugar, then the system 100 can correlate such non-clinical and clinical data 302, 304 to indicate a positive health impact for the individual. Accordingly, the system can correlate instance of a low sugar diet when the user's blood sugar is high as being good. Similarly, if the individual does not have a high sugar diet and the clinical data 304 shows the individual has low blood sugar, then the health impact of a low sugar diet when the user's blood sugar is low is neutral, i.e., low sugar diets when blood sugar is low is not required.

As illustrated, by correlating an individual's non-clinical data 302 with the individual's clinical data 304, the precision patient health platform 116 can provide the subject individual with insights relating to how the behavior, including the tendencies and/or lifestyle choices, of the individual can impact the health of the individual.

FIG. 3B provides an illustration of contextual data that can be derived from consideration of non-clinical data providing additional information that alters diet health impact. For example, when a patient has a high-sugar diet even without high blood sugar, a diabetes diagnosis significantly changes the health impact of the diet from neutral to very negative. More specifically, the example shown in FIG. 313 illustrates a block diagram 350 depicting an exemplary correlations involving non-clinical data 352, clinical data 354, and diagnosis data 356 according to certain exemplary embodiments. As shown, in some embodiments, for a given individual, the precision patient health platform 116 can include actual diagnosis data 356 associated with that individual. For example, the individual may already be diagnosed with one or more specific chronic diseases. In such embodiments, the deep learning module 218 can correlate such diagnosis data 356 with non-clinical data 352 and clinical data 354 to assess the health impact individual's behavior, including tendencies, habits, and/or lifestyle, among other characteristics, as reflected by the non-clinical data, has on the overall health of that individual.

Continuing with the above example, the non-clinical data 352 can, in this example, correspond to a risk factor relating to whether the diet of the individual is high in sugar, and the clinical data 354 can correspond to a measurement or analysis of the individual of a similar, or related risk factor, as the non-clinical data 352, and, moreover, may not be ancillary to the non-clinical data 325. Thus, in this example, as the non-clinical data 352 is related to a high sugar diet, the clinical data can related to whether testing of a blood sample from the individual indicates the individual has high blood sugar. The diagnosis data 356 can also correspond to, or otherwise be related in to, the risk, factors being identified by the non-clinical data 352 and the clinical data 354, as well as correspond to an actual medical diagnosis of the user. For example, as the non-clinical and clinical data 352, 354 are related to sugar consumption and blood sugar level, respectively, the diagnosis data 356 can also be sugar related, including, for example, whether the individual has been diagnosed as having diabetes.

As shown in FIG. 313, if the subject individual has a high sugar diet (based on their non-clinical data 352), and the individual's lab tests show high blood sugar, as indicated by the clinical data 354, and the individual is diagnosed with diabetes, then the health impact of a high sugar diet when the user's blood sugar is high and the user is diagnosed with diabetes is very negative, i.e., high sugar diets when blood sugar is high and the individual has diabetes is very bad. As also indicated by the correlations shown in FIG. 38, if the non-clinical data 352 indicates the individual has a high sugar diet, and the clinical data 354 indicates that individual's lab tests show high blood sugar level, and the individual is not diagnosed with diabetes, then the health impact of a high sugar diet when the individual's blood sugar is high, and the individual is not diagnosed with diabetes is negative, i.e., high sugar diets when blood sugar is high but the individual is not diagnosed with diabetes is had.

If the individual has a high sugar diet (based on their non-clinical data), and the individual's lab tests do not show high blood sugar, and the individual is diagnosed with diabetes, then the health impact of a high sugar diet, when the individual's blood sugar is not high, and the individual is diagnosed with diabetes is very negative, i.e., high sugar diets when blood sugar is not high and you have diabetes is had. Hate individual has a high sugar diet, and the individual's lab tests do not show high blood sugar, and the individual is not diagnosed with diabetes, then the health impact of a high sugar diet when the individual's blood sugar is not high, and the individual is not diagnosed with diabetes is neutral, i.e., high sugar diets when blood sugar is not high but the individual is not diagnosed with diabetes is fine.

If the individual does not have a high sugar diet (based on their non-clinical data), and the individual's lab tests show high blood sugar, and the individual is diagnosed with diabetes, then the health impact of not having a high sugar diet, when the individual's blood sugar is high, and the individual is diagnosed with diabetes is very positive, i.e., low sugar diets when blood sugar is high and you have diabetes is very good. If the individual does not have a high sugar diet, and the individual's lab tests show high blood sugar, and the individual is not diagnosed with diabetes, then the health impact of a low sugar diet when, the individual's blood sugar is high, and the individual is not diagnosed with diabetes is positive, i.e., low sugar diets when blood sugar is high hut the individual is not diagnosed with diabetes is good.

If the individual does not have a high sugar diet (based on their non-clinical data), and the individual's lab tests do not show high blood sugar, and the individual is diagnosed with diabetes, then the health impact of not having a high sugar diet, when the individual's blood sugar is not high, and the individual is diagnosed with diabetes is very negative, i.e., low sugar diets when blood sugar is not high and you have diabetes is very negative. If the individual does not have a high sugar diet, and the individual's lab tests do not show high blood sugar, and the individual is not diagnosed with diabetes, then the health impact of a low sugar diet when the individual's blood sugar is not high, and the individual is not diagnosed with diabetes is neutral.

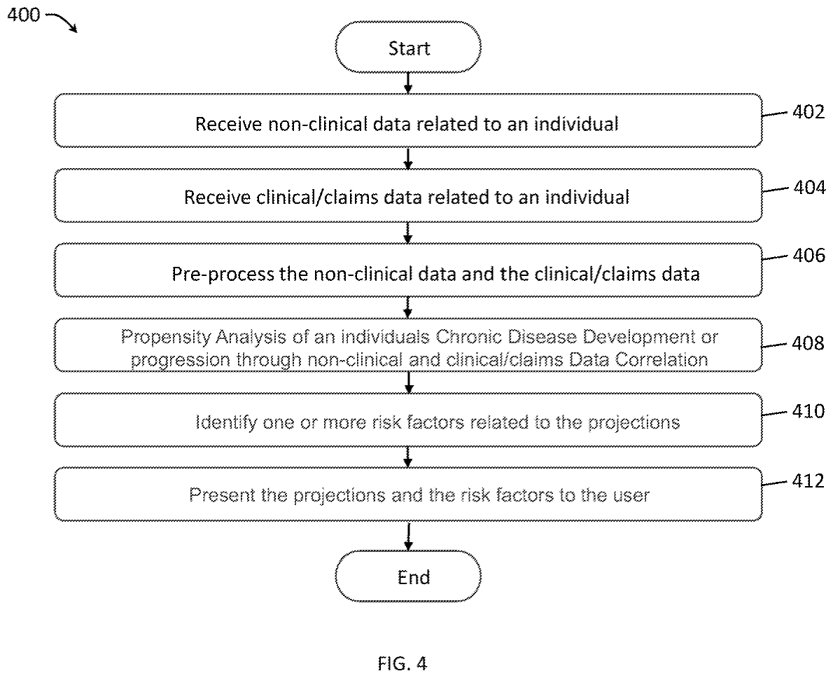

FIG. 4 illustrates a flow diagram of a method 400 of projecting an impact of individual behavior on their overall health, according to example embodiments. The method 400 is described below in the context of being carried out by the illustrated exemplary system 100 and/or the precision patient health platform 116. However, it should be appreciated that method 400 can likewise be carried out by any of the other described implementations, as well as variations thereof. Further, the method 400 corresponds to, or is otherwise associated with, performance of the blocks described below in the illustrative sequence of FIG. 4. It should be appreciated, however, that the method 400 can be performed in one or more sequences different from the illustrative sequence. Additionally, one or more of the blocks mentioned below may not be performed, and the method 400 can include steps or processes other than those discussed below.

Method 400 may begin at step 402, wherein the back-end computing system 104, including for example, the precision patient health platform. 116, can receive or retrieve, such as, for example the network 105, non-clinical data associated with one or more individuals. As discussed above, categories of non-clinical data can include travel information, event information, and purchase information. Additionally the categories of non-clinical data can be further be provided by sub-categories. For example, the category of non-clinical data can comprise a plurality of subcategories, such as, for example, subcategories related to apparel purchases, food purchases, event purchases, and/or food purchase, among others. Further, each category or subcategory can compromise a plurality of related the data points or risk factors. For example, the category of travel can include data points or risk factors related to quantity, duration, and/or location of travels related to business, and similar information for information regarding travel for leisure or vacation. Further, the category of event purchases can include data points or risk factors related to purchases related to movie theaters, which can, therefore be used to derive or predict purchases by the individual of items associated with movie going, including, for example, sugary carbonated beverages, buttered popcorn, and/or high sugary content snacks. The subcategories of apparel purchases can provide information regarding clothing sizes (e.g. purchases of men's big and tall apparel may indicate at least potential obesity) and food purchases can provide information relating to data points or other risk factors relating to dietary or nutritional consumption food, including with respect to purchases from fast food restaurants or vegan restaurants, among others. Such non-clinical data that is received at step 402 can further include a variety of other data, including, for example, habitual data (e.g., alcohol consumption, smoking, drug consumption, physical activity, etc.) and/or routine data (e.g., medicate refill frequency, check-up frequency, etc.), among other data.

At step 404, the hack-end computing system 104 can receive or retrieve, such as, for example, via the network 105, clinical data related to one or more individuals. In some embodiments, the precision patient health platform 116 can receive or retrieve clinical data, including, for example, healthcare data, associated with a plurality of individuals from one or more healthcare systems 106. Exemplary clinical data can include, but is not limited to, background data (e.g., family health history, age, gender, ethnic background, etc.), medication data (e.g., statins, low-dose aspirin, anti-anxiety, etc.), and patient specific data (e.g., body mass index (BMI), weight, blood pressure, cholesterol, clinical diagnoses (e.g., diabetes, anemia, etc.)), among other data.

At step 406, the back-end computing system 104 can preprocess the non-clinical data and the clinical data. In some embodiments, in preprocessing the non-clinical data and the clinical data, precision patient health platform 116 can perform an encoding operation, such as, for example, via use of the encoding module 211, and/or a transformation operation, such as, for example, via use of the transform module 214. For example, the encoding module 212 can encode the input data (e.g., the clinical data and the non-clinical data) into a standardized format, as previously discussed. As also discussed above, such encoding of the input data can assist in downstream processing of the input data. For example, in some embodiments, the encoding module 212 can encode or transform the clinical data and non-clinical data into a customized data format and/or an industry standard data format, including for example, a unified Fast Healthcare Interoperability Resources (FHIR®) standard, among other structured data formats.

As previously discussed, during the transformation phase, the transform module 214 can distill the non-clinical data and the clinical data down from a large feature space, or large collection of data points, to a smaller feature space, or smaller collection of data points. Moreover, the transform module 214 can perform a distillation process in which transform module 214 consolidate such health codes into higher order semantic categories, as also discussed above.

At step 408, the back-end computing system 104 can project an impact the behavior, including, for example, habits, tendencies, and lifestyle, among other factors as indicated by the non-clinical data, of an individual has on the health of that individual. For example, as discussed above, given the encoded and transformed non-clinical and clinical data, the deep learning module 218 can correlate the non-clinical data with the clinical data to determine a propensity of the subject individual to have and/or develop one or more chronic diseases, such as, for example, cardiovascular disease, dementia, and/or diabetes, among other chronic diseases. Further, at step 408, to the extent the individual does not have, or has not been identified as having, a chronic disease, or a particular chronic disease, the clustering module 216 can be used by the platform 116 to identify whether the risk profile of that individual, such as, for example, the non-clinical risk profile, does, or does not, cluster or can be grouped with other individuals that do have a chronic disease and/or to identify type(s) of chronic disease(s) of those other individuals who have similar risk profiles.

At step 410, the back-end computing system 104 can also identify one or more risk factors related the projected clinical data, non-clinical data, and or the corresponding risk profile(s). For example, the deep learning module 218 of the precision patient health platform 116 can identify those risk factors that are either negatively or positively correlated with a given chronic disease, as discussed above. In some embodiments, the insights module 220 can utilize Shapley value regression techniques to determine the importance of each risk factor that can contribute to the output generated by deep learning module 218, as also discussed above.

At step 412, the back-end computing system 104, including the insights layer 206 and one or more of the associated modules 224, 226, 228, 230 of the precision patient health platform 116 can present the information generated or otherwise outputted by the modeling layer 204 to the individual. For example, according to certain embodiments, the precision patient health platform 116 can generate a portal, accessible to the individual via the precision patient health platform device 102, in which the individual can access the projections generated by deep learning module 218. For example, the insights layer 206 can be utilized to generate a histogram of the predicted probabilities of chronic diseases for a population of individuals.

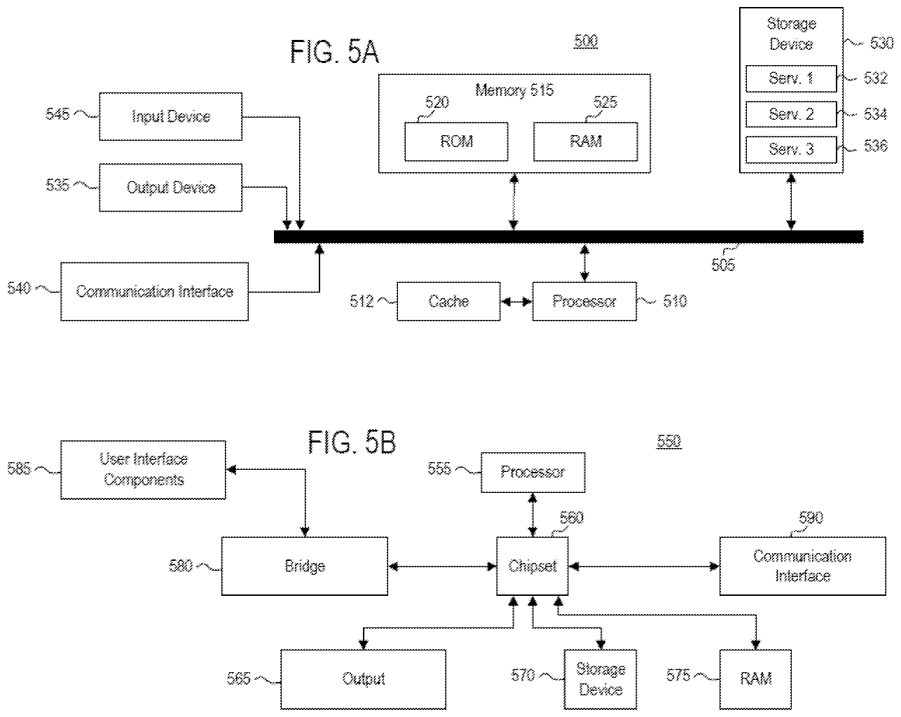

FIG. 5A illustrates a system bus architecture of a computing system 500, according to certain exemplary embodiments. The system 500 can be representative of at least a portion of the precision patient health platform device 102 and/or the back-end computing system 104. One or more components of the system 500 can be in electrical communication with each other using a bus 505. The system 500 can include a processing unit WRY or processor) 510 and a system bus 505 that couples various system components, including, for example, a system memory 515, such as, for example, a read only memory (ROM) 520 and a random access memory (RAM) 525, to one or more processors 510. The system 500 can also include a cache of high speed memory 515 that can be connected directly with, in close proximity to, or integrated as part of the processor 510. Additionally, the system 500 can copy data from the memory 515 and/or a storage device 530 to a cache 512 for quick access by the processor 510. In this way, the cache 512 can provide a performance boost that avoids processor 510 delays while waiting for data. These and other modules can control or be configured to control the processor 510 to perform various actions. Other system memory 515 can be available for use as well. The memory 515 can include multiple different types of memory with different performance characteristics. The processor 510 can include any general purpose processor and a hardware module or software module, such as service 1 532, service 2 534, and service 3 536 stored in a storage device 530, configured to control the processor 510 as well as a special-purpose processor where software instructions are incorporated into the actual processor design. The processor 510 can essentially be a completely self-contained computing system, containing multiple cores or processors, a bus, memory controller, cache, etc. A multi-core processor can be symmetric or asymmetric.

To enable user interaction with the computing system 500, an input device 545 can represent any number of input mechanisms, such as a microphone for speech, a touch-sensitive screen for gesture or graphical input, keyboard, mouse, motion input, speech and so forth. An output device 535 can also be one or more of a number of output mechanisms known to those of skill in the art. In some instances, multimodal systems can enable an individual to provide multiple types of input to communicate with the computing system 500. A communications interface 540 can generally govern and manage the user input and system output. There is no restriction on operating on any particular hardware arrangement and therefore the basic features here may easily be substituted for improved hardware or firmware arrangements as they are developed.

The storage device 530 can be a non-volatile memory and can be a hard disk or other types of computer readable media which can store data that is accessible by a computer, such as magnetic cassettes, flash memory cards, solid state memory devices, digital versatile disks, cartridges, random access memories (RAMs) 525, and/or read only memory (ROM) 520, as well as hybrids thereof.

The storage device 530 can include services 532, 534, 536 for controlling the processor 510. Other hardware or software modules are also contemplated. The storage device 530 can be connected to the system bus 505. In one aspect, a hardware module that performs a particular function can include one or more software components stored in one or more computer-readable mediums in connection with the necessary hardware components, such as the processor 510, bus 505, output device 535 (e.g., display), and so forth, to carry out an associated function,

FIG. 5B illustrates a computer system 550 having a chipset architecture that can represent at least a portion of the precision patient health platform device 102 and/or the back-end computing system 104. Computer system 550 may be an example of computer hardware, software, and firmware that may be used to implement the disclosed technology. The system 550 can include one or more processors 555, representative of any number of physically and/or logically distinct resources capable of executing software, firmware, and hardware configured to perform identified computations. The processor 555 can communicate with a chipset 560 that can control input to and output from the processor 555. In this example, a ellipse 560 can output information to an output 565, such as, for example, a display, and can read and write information to a storage device. 570, which can include magnetic media, and solid state media, for example. The chipset 560 can also read data from, and write data to, a storage device 575 (e.g., RAM). A bridge 580 for interfacing with a variety of user interface components 585 can be provided for interfacing with the chipset 560. Such a user interface components 585 can include a keyboard, a microphone, touch detection and processing circuitry, and/or a pointing device, such as, for example, a mouse, and so on. In general, inputs to the system 550 can come from any of a variety of sources, including machine generated and/or human generated.

The ellipse. 560 can also interface with one or more communication interfaces 590 that can have different physical interfaces. Such communication interfaces can include interfaces for wired and wireless local area networks, for broadband wireless networks, as well as personal area networks. Some applications of the methods for generating, displaying, and using the GUI 600, 700 disclosed herein can include receiving ordered datasets over the physical interface or be generated by the machine itself by the processor 555 analyzing data stored in the storage device 570 or the storage device 575. Further, the machine may receive inputs from a user through user interface components 585 and execute appropriate functions, such as, for example, browsing functions by interpreting these inputs using the processor 555.

It may be appreciated that the exemplary systems 500, 550 discussed herein can have more than one processor 510, or be part of a group or cluster of computing devices networked together to provide greater processing capability.

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A method for determining a propensity of an individual to have or develop one or more chronic diseases and output the determined propensity fear display on a precision patient health platform device, the method comprising:
receiving, by a back-end system from one or more third party servers, non-clinical data related to an individual;
receiving, by the back-end system from one or more healthcare systems, clinical data related to the individual;
pre-processing, by the hack-end system, the non-clinical data and the clinical data;
projecting, by the hack-end system, the propensity of the individual for developing at least one chronic disease of the one or more chronic diseases by correlating the non-clinical data with the clinical data;
identifying, by the back-end system, for the at least one chronic disease, at least one non-clinical risk factor that positively or negatively affects the propensity of the individual for developing the at least one chronic disease; and
generating, by the back-end system, a portal comprising a projection of the propensity of the individual to develop the at least one chronic disease.

2. The method of claim 1, wherein pre-processing of the non-clinical data and the clinical data comprises:
encoding the non-clinical data and the clinical data into a standardized format.

3. The method of claim 1, further comprising:
receiving, by the back-end system, at least one secondary guideline related to the one or more chronic diseases; and
generating, by the back-end system, a risk profile corresponding to each chronic disease of the one or more chronic diseases, wherein the risk profile comprises a first set of risk factors that negatively correspond to each chronic disease and a second set of risk factors that positively correspond to each chronic disease, and
wherein at least a portion of the first set of risk factors and another portion of the second set of risk factors are derived from information distilled by the back end system from the secondary guideline.

4. The method of claim 3, wherein pre-processing the non-clinical data and the clinical data comprises:
distilling, by the hack-end system, the non-clinical data down to a first set of non-clinical data point for the first set of risk factors and a second set of non-clinical data points for the second set of risk factors; and
distilling, by the back-end system, the clinical data down to a first set of clinical data points for the first set of risk factors and a second set of data points for the second set of risk factors.

5. The method of claim 4, wherein projecting the propensity of the individual for having or developing the at least one chronic disease by correlating the non-clinical data with the clinical data comprises correlating the distilled non-clinical data with the distilled clinical data.

6. The method of claim 1, wherein identifying, for the at least one chronic disease, the at least one non-clinical risk factor that positively or negatively affects the propensity of the individual for the at least one chronic disease comprises:
applying, by the back-end system, a Shapley value regression technique to determine an importance of each of a plurality of non-clinical risk factors to the propensity of the individual for that chronic disease.

7. The method of claim 1, further including correlating the non-clinical and the clinical data comprises identifying a statistical relationship between the non-clinical data and the clinical data, and
wherein the clinical data comprises diagnosis data for the individual.

8. A non-transitory computer readable medium comprising one or more sequences of instructions, which, when executed by a processor, causes a computing system to perform operations comprising:
receiving, from one or more third party servers, a non-clinical data related to an individual;
receiving, from one or more healthcare systems, a clinical data related to the individual;
pre-processing the non-clinical data and the clinical data;
projecting a propensity of the individual for developing a chronic disease by correlating the non-clinical data with the clinical data;
identifying non-clinical factors that positively or negatively affect the propensity of the individual for the chronic disease; and
generating a portal comprising a projection of the propensity of the individual to develop the chronic disease.

9. The non-transitory computer readable medium of claim 8, wherein pre-processing, by the computing system, the non-clinical data and the clinical data comprises:
encoding the non-clinical data and the clinical data into a standardized format.

10. The non-transitory computer readable medium of claim 8, further comprising:
receiving a secondary guideline related to of the chronic disease; and
generating a risk profile corresponding to the chronic disease, wherein the risk profile comprises a first set of factors that negatively correspond to the chronic disease and a second set of factors that positively correspond to the chronic disease, and
wherein at least a portion of the first set of factors and another portion of the second set of factors are derived from information distilled from the secondary guideline.

11. The non-transitory computer readable medium of claim 10, wherein pre-processing, by the computing system, the non-clinical data and the clinical data comprises:
distilling the non-clinical data down to the first set of factors and the second set of factors; and
distilling, the clinical data down to the first set of factors and the second set of factors.

12. The non-transitory computer readable medium of claim 11, wherein projecting the propensity of the individual for the chronic disease by correlating the non-clinical data with the clinical data comprises:
correlating the distilled non-clinical data with the distilled clinical data by identifying a statistical relationship between the non-clinical data and the clinical data.

13. The non-transitory computer readable medium of claim 8, wherein identifying the non-clinical factors that positively or negatively affect the propensity of the individual for the chronic disease comprises:
applying a Shapley value regression technique to determine an importance of each of the factors to the propensity of the individual to develop the chronic disease.

14. The non-transitory computer readable medium of claim 8, wherein the clinical data comprises diagnosis data for the individual.

15. A system comprising:
a processor; and
a memory having programming instructions stored thereon, which, when executed by the processor, causes the system to perform operations comprising:
receiving, from one or more third party servers, non-clinical data related to an individual;
receiving, from one or more healthcare systems, clinical data related to the individual;
pre-processing the non-clinical data and the clinical data;
projecting a propensity of the individual for developing at least one chronic disease of a plurality of chronic diseases by correlating the non-clinical data with the clinical data;
identifying for each chronic diseases of the plurality of chronic diseases, non-clinical that positively or negatively affect the propensity of the individual for that chronic disease; and
generating a portal comprising projections for the individual developing the at least one chronic disease.

16. The system of claim 15, wherein pre-processing the non-clinical data and the clinical data comprises:
encoding the non-clinical data and the clinical data into a standardized format.

17. The system of claim 15, wherein the operations further comprise:
receiving a plurality of secondary guidelines related to the plurality of chronic diseases; and
generating a risk profile corresponding to each chronic disease of the plurality of chronic diseases, wherein the risk profile comprises a first set of factors that negatively correspond to each chronic disease and a second set of factors that positively correspond to each chronic disease. And
wherein at least some of both the first set of factors and the second set of factors are derived from information distilled from the plurality of secondary guidelines.

18. The system of claim 17, wherein pre-processing the non-clinical data and the clinical data comprises:
distilling the non-clinical data down to the first set of factors and the second set of factors; and
distilling the clinical data down to the first set of factors and the second set of factors.

19. The system of claim 18, wherein projecting the propensity of the individual for developing at least one chronic disease of a plurality of chronic diseases by correlating the non-clinical data with the clinical data comprises:
correlating the distilled non-clinical data with the distilled clinical data by identifying a statistical relationship between the non-clinical data and the clinical data.

20. The system of claim 15, wherein identifying for each chronic disease, the non-clinical factors that positively or negatively affect the propensity of the individual for that chronic disease comprises:
applying a Shapley value regression technique to determine an importance of each non-clinical factor to the propensity of the individual for that chronic disease.
